(19) United States
(12) Patent Application Publication
(10) Pub. No.: US 2024/0194313 A1
(43) Pub. Date: Jun. 13, 2024

(54) REPORTING IN POCUS WORKFLOWS

(71) Applicant: FUJIFILM SONOSITE, INC., Bothell, WA (US)

(72) Inventors: David Knapp, Bellevue, WA (US); Wendy Swan, Bothell, WA (US); Craig Chamberlain, Seattle, WA (US); Christopher Bartholomew, Bothell, WA (US)

(21) Appl. No.: 18/080,499

(22) Filed: Dec. 13, 2022

(51) Int. Cl. G16H 15/00; G16H 10/20; G16H 40/40; G16H 50/20; G16H 70/20

(52) U.S. Cl. CPC G16H 15/00; G16H 10/20; G16H 40/40; G16H 50/20; G16H 70/20

(57) ABSTRACT

Systems and methods for automated reporting in Point-of-Care ultrasound (POCUS) workflows are described. In some embodiments, an ultrasound reporting system includes a memory configured to maintain a mapping of system events to worksheet answers. A processor system is coupled to the memory and is configured to implement a reporting application at least partially in hardware. The reporting application is implemented to determine, during an ultrasound examination, an occurrence of a system event of the system events. The reporting application is also implemented to determine, based on the mapping, a worksheet answer of the worksheet answers that is mapped to the system event. The reporting application is also implemented to populate, during the ultrasound examination and responsive to the determination of the occurrence of the system event, a medical worksheet with the worksheet answer.

Embodiments disclosed herein relate to ultrasound systems. More specifically, embodiments disclosed herein relate to automated reporting in ultrasound workflows.

### BACKGROUND

Generally, Point-of-Care ultrasound (POCUS) departments establish standards of care to deliver consistent outcomes, ensure that proper ultrasound training occurs and the department receives adequate billing for performed procedures. These standards of care improve overall patient quality of care as well as improve the credibility of the POCUS department within an institution that can include radiology, surgery, and other departments.

Typically, a primary mechanism that POCUS Departments use to establish these standards of care is for clinicians to submit reports for each ultrasound study that is performed. These reports are referred to as worksheets by the American College of Emergency Physicians. Generally, worksheets submitted are used for billing, quality control, and accreditation.

Generally, protocols are used to ensure a department compliance for standards of care to achieve a given diagnostic goal. A protocol can be described as a breakdown of a workflow into steps, generally equating to specific views, that are intended to be completed to comply with a specific standard of care. Some example protocols include focused assessment with sonograph (FAST), eFAST, bedside lung ultrasound in emergency (BLUE), and focus assessed transthoracic echo (FATE). Protocols are also used in musculoskeletal (MSK) ultrasound to expound upon a lengthy set of views for complete exams.

However, documenting ultrasound studies in a POCUS environment takes time away from other clinical duties. POCUS physicians often document studies asynchronously with the ultrasound examination, such as during small breaks during the day, batching up several studies from a given time period, or at the end of the day, entering examination results for the day's examinations. Hence, data may not be properly entered into the worksheet, or incorrect data can be entered, because the ultrasound examination is not fresh in the physician's mind when they are populating the worksheet. An alternative to entering worksheets asynchronously is for the worksheets to be entered at the bedside through worksheets on the ultrasound system. However, integrating these worksheets into a seamless workflow within an ultrasound system has proven to be difficult from a user experience perspective, and can result in wasted time for the physician and excessive examination time for the patient.

### SUMMARY

Systems and methods for automated reporting in Point-of-Care ultrasound (POCUS) workflows are described. In some embodiments, an ultrasound reporting system includes a memory configured to maintain a mapping of system events to worksheet answers. A processor system is coupled to the memory and is configured to implement a reporting application at least partially in hardware. The reporting application is implemented to determine, during an ultrasound examination, an occurrence of a system event of the system events. The reporting application is also implemented to determine, based on the mapping, a worksheet answer of the worksheet answers that is mapped to the system event. The reporting application is also implemented to populate, during the ultrasound examination and responsive to the determination of the occurrence of the system event, a medical worksheet with the worksheet answer.

In some embodiments, an ultrasound reporting system includes a processing system and at least one computer-readable storage medium configured to store instructions executable via the processing system to implement a reporting application. The reporting application is configured to determine, during an ultrasound examination, a satisfaction of a threshold condition. The reporting application is also configured to determine a question on a medical worksheet corresponding to the threshold condition and to populate, during the ultrasound examination and responsive to the determination of the satisfaction of the threshold condition, an answer field of the question on the medical worksheet corresponding to the threshold condition.

In some embodiments, an ultrasound reporting system includes a processing system and at least one computer-readable storage medium that is configured to store instructions executable via the processing system to implement a reporting application. The reporting application is configured to access a dictionary of questions for an ultrasound examination and to determine an occurrence of a system event during the ultrasound examination. The reporting application is also configured to generate, based on the occurrence of the system event, a clinical result that includes an answer to a question in the dictionary.

In some embodiments, a method implemented by a computing device includes receiving a user-defined mapping of a system event to a worksheet answer and determining an occurrence of the system event during an ultrasound examination. The method also includes populating, automatically and responsive to determining of the occurrence of the system event, a medical worksheet with the worksheet answer.

In some embodiments, a method implemented by a computing device includes determining, during an ultrasound examination, a satisfaction of a threshold condition, and determining a question on a medical worksheet corresponding to the threshold condition. The method also includes populating, automatically and responsive to the determining the question, an answer field of the question on the medical worksheet based on the satisfaction of the threshold condition.

In some embodiments, a method implemented by a computing device includes determining an anatomy being imaged, with a neural network implemented at least partially in hardware of the computing device. The method also includes displaying, automatically and based on the anatomy, a protocol step that includes a question about the anatomy.

In some embodiments, a method implemented by a computing device includes determining an operator identification for an operator of the computing device during an ultrasound examination, and determining, based on the operator identification, an order for a protocol. The method also includes displaying, during the ultrasound examination, steps of the protocol in the order.

In some embodiments, a method implemented by a computing device includes accessing a dictionary of questions for an ultrasound examination, and determining an occurrence of a system event during the ultrasound examination. The method also includes generating, based on the occurrence of the system event, a clinical result that includes an answer to a question in the dictionary.

Other systems, devices, and methods for reporting in POCUS workflows are also described.

### BRIEF DESCRIPTION OF THE DRAWINGS

The appended drawings illustrate examples and are, therefore, exemplary embodiments and not considered to be limiting in scope.

FIG. 1 illustrates some embodiments of an ultrasound transducer probe having an ultrasound transducer assembly.

FIG. 2A is a view illustrating a sample protocol workflow without assisted reporting implemented by an ultrasound system according to some embodiments.

FIG. 2B is a view illustrating a sample protocol workflow with assisted reporting implemented by an ultrasound system according to some embodiments.

FIG. 3 is a view illustrating an example interface for a protocol self-report step implemented by an ultrasound system according to some embodiments.

FIG. 4 is a view illustrating an example interface for a protocol self-report step implemented by an ultrasound system according to some embodiments.

FIG. 5 illustrates a data flow diagram of a process implemented by an ultrasound reporting system according to some embodiments.

FIG. 6 illustrates a data flow diagram of a process implemented by an ultrasound reporting system according to some embodiments.

FIG. 7 illustrates a data flow diagram of a process implemented by an ultrasound reporting system according to some embodiments.

FIG. 8 illustrates a data flow diagram of a method implemented by a computing device according to some embodiments.

FIG. 9 illustrates a data flow diagram of a method implemented by a computing device according to some embodiments.

FIG. 10 illustrates a data flow diagram of a method implemented by a computing device according to some embodiments.

FIG. 11A illustrates a data flow diagram of a method implemented by a computing device according to some embodiments.

FIG. 11B illustrates a data flow diagram of a method implemented by a computing device according to some embodiments.

FIG. 12 illustrates a block diagram of an example computing device that can perform one or more of the operations described herein, in accordance with some embodiments.

### DETAILED DESCRIPTION

In the following description, numerous details are set forth to provide a more thorough explanation of the present invention. It will be apparent, however, to one skilled in the art, that the present invention may be practiced without these specific details. In other instances, well-known structures and devices are shown in block diagram form, rather than in detail, in order to avoid obscuring the present invention.

Systems, methods, and devices for automated and/or assisted reporting in POCUS workflows are described. In some embodiments, an ultrasound reporting system includes a memory configured to maintain a mapping of system events to worksheet answers and a processor system coupled to the memory and configured to implement a reporting application at least partially in hardware. The reporting application determines an occurrence of one of the system events during an ultrasound examination. The reporting application determines, based on the mapping, a worksheet answer of the worksheet answers that is mapped to that system event. The reporting application populates a medical worksheet with the worksheet answer during the ultrasound examination in response to determining the occurrence of the system event.

Generally, the term “worksheet” is used herein to refer to a report in which clinical results are recorded, and includes questions and answers per a protocol. Although described with respect to reporting clinical results to a worksheet, embodiments disclosed herein are not so limited. Rather, embodiments disclosed herein can generate a clinical result and populate it to any suitable structure, such as a worksheet, a file (e.g., a flat file), an application programming interface (API) call, a database, and the like, that can be further processed to affect reporting.

The following describes an ultrasound system that can be used with automated and/or assisted data entry, including, for example, automated and/or assisted data entry for workflows that following one or more established protocols.

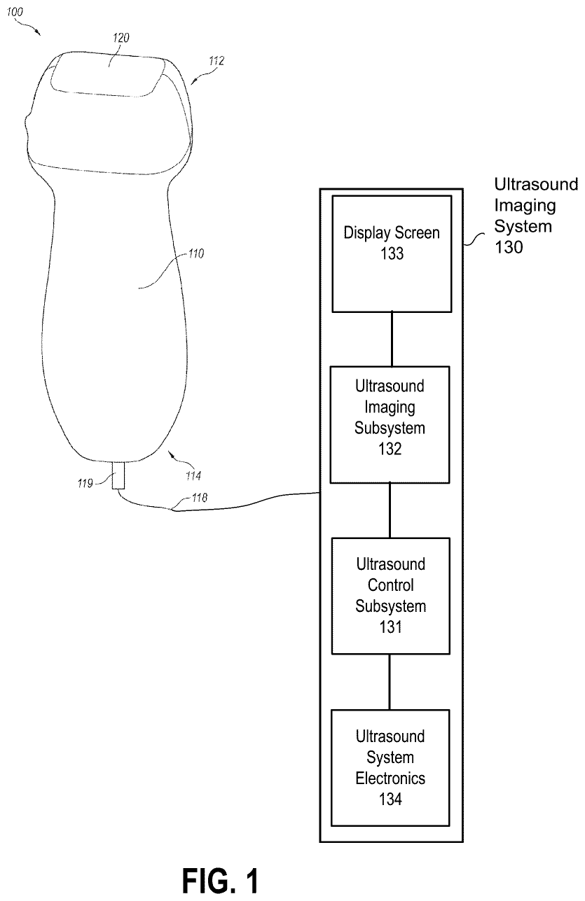

FIG. 1 illustrates an ultrasound system 100 including a transducer probe having an ultrasound transducer assembly configured in accordance with an embodiment of the disclosed technology. Referring to FIG. 1, an ultrasound system 100 includes an ultrasound transducer probe 110 that includes an enclosure extending between a distal end portion 112 and a proximal end portion 114. The ultrasound transducer probe 110 is electrically coupled to an ultrasound imaging system 130 that includes ultrasound control subsystem 131, ultrasound imaging subsystem 132, display screen 133, and ultrasound system electronics 134. The transducer probe 100 can be electrically coupled to the ultrasound imaging system 130 via a cable 118 that is attached to the proximal end of the probe by a strain relief element 119. Additionally or alternatively, the ultrasound probe 100 can be electrically coupled to the ultrasound imaging system 130 via a wireless communication link.

A transducer assembly 120 having one or more transducer elements is electrically coupled to the ultrasound system electronics 134 in ultrasound imaging system 130. In operation, transducer assembly 120 transmits ultrasound energy from the one or more transducer elements toward a subject and receives ultrasound echoes from the subject. The ultrasound echoes are converted into electrical signals by the one or more transducer elements and electrically transmitted to the ultrasound system electronics 134 in ultrasound imaging system 130 to form one or more ultrasound images.

Capturing ultrasound data from a subject using an exemplary transducer assembly (e.g., the transducer assembly 120) generally includes generating ultrasound, transmitting ultrasound into the subject, and receiving ultrasound reflected by the subject. A wide range of frequencies of ultrasound can be used to capture ultrasound data, such as, for example, low frequency ultrasound (e.g., less than 15 MHz) and/or high frequency ultrasound (e.g., greater than or equal to 15 MHz) can be used. Those of ordinary skill in the art can readily determine which frequency range to use based on factors such as, for example, but not limited to, depth of imaging and/or desired resolution.

In some embodiments, ultrasound imaging system 130 includes ultrasound system electronics 134 that can include one or more processors, integrated circuits, ASICs, FPGAs, power sources, and the like to support the functioning of ultrasound imaging system 130 in a manner well-known in the art. In some embodiments, ultrasound imaging system 130 also includes ultrasound control subsystem 131 having one or more processors. At least one processor can cause electrical signals to be sent to the transducer(s) of probe 100 to emit sound waves and also receive the electrical pulses from the probe that were created from the returning echoes. One or more processors can process the raw data associated with the received electrical pulses and form an image that is sent to ultrasound imaging subsystem 132, which can display the image on display screen 133. Thus, display screen 133 can display ultrasound images from the ultrasound data processed by the processor of ultrasound control subsystem 131. Additionally or alternatively, the display screen 133 can display a medical worksheet in part or in whole.

In some embodiments, the ultrasound imaging system 130 also has one or more user input devices (e.g., a keyboard, a cursor control device, etc.) that input data and allow the taking of measurements from the display of the ultrasound display subsystem. The ultrasound imaging system 130 can include one or more output devices, such as a disk storage device (e.g., hard, floppy, thumb drive, compact disks (CD), digital video discs (DVDs)) for storing the acquired images, and a printer that prints the image from the displayed data. These input and output devices have not been shown in FIG. 1 to avoid obscuring the techniques disclosed herein.

In some embodiments, the ultrasound transducer assembly 120 includes a transducer layer configured to emit ultrasound energy, one or more matching layers overlaying the transducer layer, a thermally conductive layer overlaying the one or more matching layers, and a lens overlaying the thermally conductive layer. In some embodiments, the lens is a compound acoustic lens for an ultrasound transducer assembly having an ultra-high frequency phase array. In such a case, in some embodiments, the processor-based system coupled to the memory and the ultrasound probe, upon execution of the instructions, causes the ultrasound probe to transmit an ultrasound beam through the compound acoustic lens that focuses the ultrasound beam.

### Automated Data Entry

In some embodiments, an ultrasound system performs automated and/or assisted data entry of POCUS worksheet data using a protocol solution. Typically, POCUS reporting is used to comply with standards of care in electronic medical record (EMR) departments. These reports are called worksheets by American College of Emergency Physicians (ACEP). Worksheet-based reporting ensures consistent outcomes, training, billing, and generating department metrics. For conventional systems, worksheet data entry is a logistical challenge. Some departments prefer “bedside worksheet entry” as opposed to entering data into worksheets asynchronously via computer workstations, e.g., after an examination, such as at the end of the day. For bedside worksheet data entry, data is are entered during the exam setting, helping to resolve any memory issues associated with asynchronous data entry, and streamlining the workflow. Protocols are used to ensure department compliance with standards of care. Protocols breakdown a clinical workflow into steps, such as by specifying which views are required for compliance. A sample of a protocol set of features may include the following:
1. Select a protocol (turn on protocol)
2. View all steps within a protocol
3. Complete protocol steps in a standardized, pre-defined order
4. Complete protocol steps out of order
5. Understand if a step is done, selected, or not done
6. Omit a step of the protocol (select image not obtainable)
7. Document the protocol by saving images/clips
8. Label the image or clip with predefined annotation labels
9. Save multiple images/clips for any protocol step
10. View thumbnails of saved image(s)/clip(s) for each step
11. Pause/resume a protocol (investigate/document outside of protocol)
12. Switch a transmitting circuitry (Tx) for a protocol step
13. Set underlying exam type for a protocol step
14. Set imaging parameters (depth, gain, dynamic range, etc.) for a given step
15. Document findings for an individual step or entire protocol
16. Enabling artificial intelligence assist features, e.g., lung sliding, free fluid, automatic ejection fraction (auto EF), etc.
17. Access LEARN module for an individual step or entire protocol
18. Optional instructional steps that specifically instructs or reminds the clinical user for the upcoming view
19. Customize protocols
   In some embodiments, protocols are customizable to support the variety of standards of care across POCUS departments. For example, protocols need flexibility to codify the POCUS department's variable standards of care. The protocols are often customized by an institution. Supporting customized protocol workflows can allow data entry into worksheet data fields within a clinical context. For example, an eFAST protocol customized for an urgent care department can include a number of images that is less than the number of images of the eFAST protocol customized for a regular care department. In some embodiments, during the customization of protocols, an author specifies which worksheet is to be filled out for a study. In an example, the author is a department head responsible for defining protocols for the department. A data dictionary can tie worksheet data to steps in the protocol via system events (e.g., a save image event) or self-report form entry, as described in further detail below.

In some embodiments, when a clinician runs through the protocol, questions are answered in context and the ultrasound system automatically updates the worksheet, reducing time at the end of the exam, as described in further detail below.

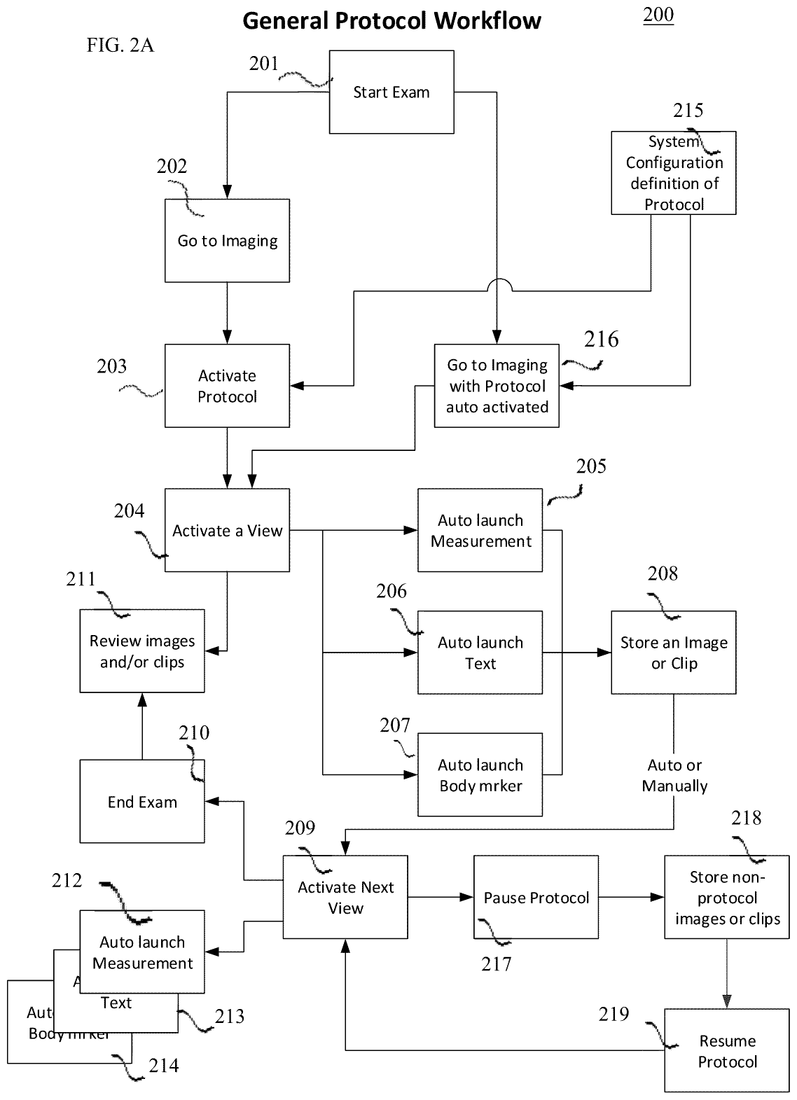

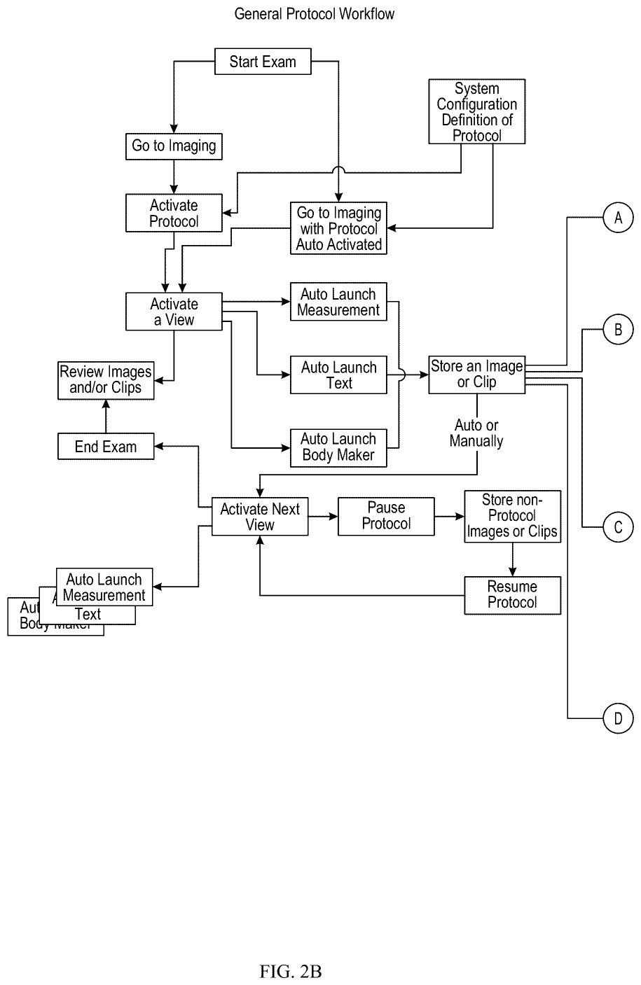

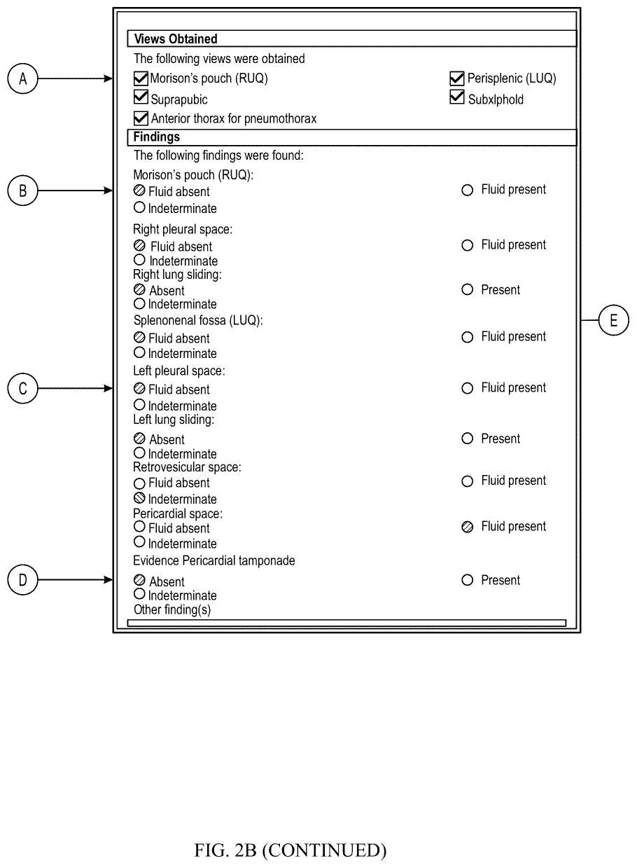

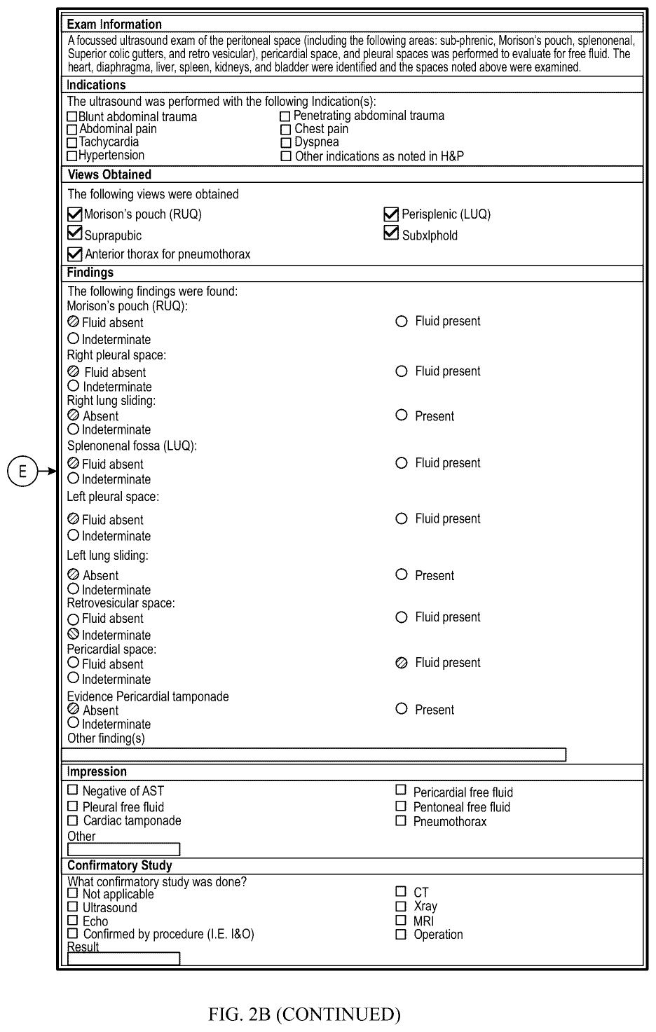

FIG. 2A is a view 200 illustrating a sample protocol workflow without assisted and/or automated reporting implemented by an ultrasound system according to some embodiments. In some embodiments, the ultrasound system includes one or more processors and a memory coupled to the one or more processors to perform at least a portion of the protocol workflow. In some embodiments, at least a portion of the general protocol workflow is performed by processing logic that can include hardware (e.g., circuitry, dedicated logic, memory, etc.), software (such as is run on a general-purpose computer system or a dedicated machine), firmware (e.g., software programmed into a read-only memory), or combinations thereof. In some embodiments, the process is performed by one or more processors of a computing device such as, for example, but not limited to, an ultrasound machine with an ultrasound imaging subsystem.

Referring to FIG. 2A, an exam is started at block 201. The sample protocol workflow goes to imaging at block 202. At block 203, a protocol is activated. At block 204, a view is activated. After the view is activated, a measurement associated with the view is automatically launched at block 205, a text associated with the view is automatically launched at block 206 and a body marker associated with the view is automatically launched at block 207. At block 208, an image or clip is stored in a memory when a view is acquired. After the image/clip is stored in the memory, a next view is activated at block 209. In some embodiments, one or more views are specified for each protocol step. In some embodiments, the next view is automatically activated. In some embodiments, the next view is manually activated.

In some embodiments, a measurement associated with the next view is automatically launched at block 212, a text associated with the next view is automatically launched at block 213 and a body marker associated with the next view is automatically launched at block 214. In some embodiments, after the next view is activated, the protocol workflow goes to block 210 where the exam ends.

In some embodiments, after the next view is activated, the protocol workflow is paused at block 217 and non-protocol images or clips are stored in a memory at block 218. After the non-protocol images or clips are stored in the memory, the protocol workflow resumes at block 219 and a next view is activated at block 209.

Embodiments described herein take advantage of each protocol step for supplying worksheet data within the context of image acquisition. These protocol steps are generally the specific views that are required as part of the documentation process. In some embodiments, data entered at a step can include:
1. System events and data may be used to automate worksheet entry. In some embodiments, this information includes information like:
   a. If an image or clip is captured, automatically documenting if a view was obtained.
2. Self-report data forms to assist a user in supplying the data required for a specific view. In some embodiments, this information includes any self-report worksheet field(s), such as:
   a. Free Fluid—Absent, Present, Indeterminate
   b. Lung Sliding—Yes/No
   c. Lung Pulse—Yes/No
   d. Lung Point—Yes/No

In the system event example, the worksheet can be automatically populated based on the occurrence of a system event (e.g., an image being saved). In the self-report example, a part of the worksheet can be displayed based on something other than a system event, such as a view selected by the operator, or a part of a protocol that is currently being performed (described below in more detail).

In some embodiments, during the customization of protocols, an author (e.g., a director in an organization responsible for defining protocols, or other author) can specify which worksheet is to be filled out for an ultrasound study. The worksheet can be from a local copy of customized worksheets. For example, a local copy of customized worksheets can be from Telexy or Fujifilm Sonosite Synchronicity, or other local copy of the customized worksheet. Each worksheet generally includes questions and answers with various types of responses that are supported (e.g., Check All that Apply, Radio Button, Short Answer, Long Answer, etc.). A data dictionary can assign reference identifiers (IDs) for specific questions and answers based on the underlying data types of the worksheet. For example, questions can be identified by Q's, e.g., Q1, Q2, Q3, etc., and answers can be indicated by sub-IDs, such as Q1_A (e.g., answer “A” to question “Q1”), Q1_B (e.g., answer “B” to question “Q1”), etc. In some embodiments, the author ties worksheet questions, e.g., worksheet questions Q1, Q2, to associated protocol steps, for example:
Q1—Was the Subcostal View Obtained?
(System event—save Image/Clip) Q1_1=Yes
Q2—Fluid Detected?

### (Self-Report) Q2_1—Absent, Q2_2—Present, Q2-3—Indeterminate

During the customization of a protocol step, the author can customize which data can be entered during that step. The user interface can display human readable values that obscure the data dictionary as described above. For example:

If configured for assisted/automated data entry into worksheets based on system events, the system can automatically enter data based on an event driven logic, e.g.,
1. On this step
2. if one or more image/clip is saved,
3. enter “Was the Subcostal View Obtained?” (Q1) as “Yes” (Q1_1)

The outcome is that the system enters the appropriate value in the worksheet when the user saves an image or clip during that step. In this example, the author of the worksheet has created a mapping between the system event of saving the image and affirmatively answering the worksheet question as to whether an appropriate view was obtained. Hence, when the system event occurs (e.g., the image is saved), the user does not need to manually enter an affirmative answer to the question “Was the Subcostal View Obtained?”. Rather, when the image is saved, the system automatically populates the worksheet with the affirmative answer to the question that is mapped to this system event.

As another example, the system can receive a user-defined mapping of a system event to a worksheet answer. The mapping can be defined by an author of a protocol (e.g., a director of a department in a care facility) and the mapping can be received by the system as part of loading a protocol onto a computing device. The mapping can also include a worksheet question answered by the worksheet answer. The system can then determine, during an ultrasound examination, the occurrence of the system event, and populate, automatically and responsive to the determination of the occurrence of the system event, a medical worksheet with the worksheet answer, thus automatically answering the question on the worksheet based on the occurrence of the system event. The system event can include the saving of an ultrasound image, and the worksheet answer can include an affirmative response as to whether the ultrasound image includes an acceptable view, as described above.

FIG. 2B is a view 220 illustrating a sample protocol workflow 221 with assisted and/or automated reporting implemented by an ultrasound system according to some embodiments. In some embodiments, the system includes one or more worksheets having an auto-populating feature enabled, such as a worksheet 222 and a worksheet 223. As shown in FIG. 2B, worksheet 221 is associated with a current step (block 224) of the protocol. As shown in FIG. 2B, at block 224 an image or clip is stored in a memory when a view is acquired. Worksheet 222 has an auto-populating feature enabled to indicate obtained views and findings. Worksheet 223 is generated based on worksheet 222. As shown in FIG. 2B, worksheet 223 is an EFAST Exam reporting worksheet that has an auto-populating feature enabled and includes obtained views and findings from worksheet 222.

In an example, the system determines a next protocol step for the ultrasound examination based on the occurrence of the system event. The system can then display a portion of the medical worksheet that corresponds to the next protocol step. In some embodiments, the system can determine an operator identification that identifies an operator conducting the ultrasound examination and determine the next protocol step based on the identification of the operator. For instance, the system can access a list of operators and their preferences, including an order in which protocol steps for a specified protocol are performed by that operator. For example, if the user prefers to start at step 3 of a protocol and then move to step 1, then the system can determine that the operator is currently at step 3, and when this step is completed, the system can display step 1 for the protocol, and/or a portion of the worksheet associated with step 1. In some embodiments, the system can determine the next protocol step based on the operator identification and history associated with this operator.

In an example, the operator identification can be used to obtain user-defined mappings for the operator (e.g., defined by the operator). In some embodiments, the system can obtain, based on the operator identification, the user-defined mapping from a database of user-defined mappings. In some embodiments, the system can also obtain the medical worksheet from the database based on the operator identification.

Additionally or alternatively, the system can determine, with a neural network implemented at least partially in hardware of a computing device (e.g., an ultrasound machine, or other computing device), that the ultrasound examination includes a step of a protocol. The system can then display, automatically and without user intervention, a portion of the medical worksheet that corresponds to the step of the protocol. In some embodiments, the system can determine that the ultrasound examination includes the step of the protocol by determining an anatomy of a patient that is being imaged.

Additionally or alternatively, in some embodiments, the system can receive a user selection of a starting point in a protocol. For instance, the system can receive the user selection via an interface displayed on the computing device, or from a database of preferences maintained for operators. The system can then configure the computing device for the ultrasound examination. In some embodiments, configuring the computing device can include displaying an interface that illustrates the protocol and the starting point, and setting an imaging parameter for the starting point, such as, for example, a gain, depth, or examination type, and the like. In an example, the interface includes a selection for an auto-populate feature to enable the auto-population of worksheet data based on mappings of system events to worksheet questions and answers (such as by auto-populating an affirmative response to a question about obtaining an acceptable view when an image is saved at block 108, as described above). In some embodiments, the operator can select the auto-populate feature in the interface to enable auto-population of worksheet data. In some embodiments, the interface can also include a selection to display a medical worksheet, or part of a medical worksheet. Hence, the system can receive a user selection of the worksheet display feature, and display the medical worksheet based on the user selection, such as by displaying a portion of the medical worksheet. The operator can then inspect the medical worksheet during the ultrasound examination to make sure that the worksheet has been properly populated.

In some embodiments, the system can auto-populate the worksheet based on a threshold condition being satisfied, such as a neural network determining that an amount of free fluid detected is greater than a threshold amount of free fluid. For instance, in some embodiments, the system can determine a satisfaction of a threshold condition during an ultrasound examination and determine a question on a medical worksheet corresponding to the threshold condition. The system can then populate, automatically and responsive to the determining the question, an answer field of the question on the medical worksheet based on the satisfaction of the threshold condition. In some embodiments, the threshold condition can include a threshold amount of free fluid, and the answer field can include an affirmation of free fluid detection. Additionally or alternatively, in some embodiments, the threshold condition can include an image quality, and the answer field can include an image quality score, an indication of an anatomy in an ultrasound image, an indication of an interventional instrument in the ultrasound image, and the like, to indicate the image quality is “good enough” to satisfy the threshold condition.

In some embodiments, the system determines the question corresponding to the threshold condition by determining that the question can be answered by the satisfaction of the threshold condition. Additionally or alternatively, in some embodiments, the system determines that the question corresponding to the threshold condition can be based on a user-defined mapping of the satisfaction of the threshold condition to the question and the answer field.

In some embodiments, the ultrasound examination includes a protocol, and determining the satisfaction of the threshold condition is performed during a current step of the protocol. In some embodiments, the system determines the question as part of the current step of the protocol. In some embodiments, the system determines the question as part of a subsequent step of the protocol.

In some embodiments, the system includes a computing device, such as an ultrasound machine, that can obtain protocols for ultrasound examinations from other computing devices, such as another ultrasound machine. For example, a first ultrasound machine can determine that it does not have protocols installed on it. For instance, the computing device can be a new ultrasound machine, or simply configured without installed protocols. The first ultrasound machine can communicate with other ultrasound machines in a care facility by querying them to determine if they have protocols installed for the care facility, or a department within the care facility. If one of the other machines answers the query to indicate that it does have protocols installed, the first ultrasound machine can obtain the protocols from the other ultrasound machine and configure itself to be ready for use with the protocols. Hence, a computing device (e.g., ultrasound machine) described herein can self-discover protocols. In some embodiments, the computing device can import protocols from other machines and export protocols to the other machines. Moreover, in some embodiments, the computing device can be updated, such as via enterprise management, to add, remove, or update protocols.

In some embodiments, the system can include a self-report feature in which a part of the worksheet is displayed based on something other than a system event, such as a view selected by the operator, or a part of a protocol that is currently being performed. For instance, the system can determine that the operator is performing a FAST examination and is looking at a right upper quadrant (RUQ), as opposed to a left upper quadrant (LUQ) or pelvis, and display questions and candidate answers to the questions that are directed to the RUQ and not the LUQ or pelvis. This self-report feature can be included as part of the protocol customization in a similar manner as the event-driven optimization previously discussed.

If the self-report feature is enabled, in some embodiments, the system can display

form controls for the user to self-report outcomes, e.g.,
On this step
Display the question and candidate answers:
Fluid Detected? (Q2)
   Absent (Q2_1)
   Present (Q2_2)
   Indeterminate (Q2_3)

In this case, the outcome would be, if the user enters Fluid Detected Absent (e.g., the user selects Q2_1 as the answer to Q2), then the system would enter that answer into the appropriate field in the worksheet. In this example, the system can display an interface with worksheet questions and candidate answers to the questions, without explicitly displaying the worksheet. When the user selects an answer via the interface, the system can populate the worksheet with the answer. In some embodiments, several self-report questions can be added to the same view.

In some embodiments, a preview feature assists the author in testing the integration of the desired data collection at the given step. In an example, the preview feature provides an indication that the user must approve the data at a given step for the data to be entered into a worksheet, such as with an “approve”, “submit”, or “next” button.

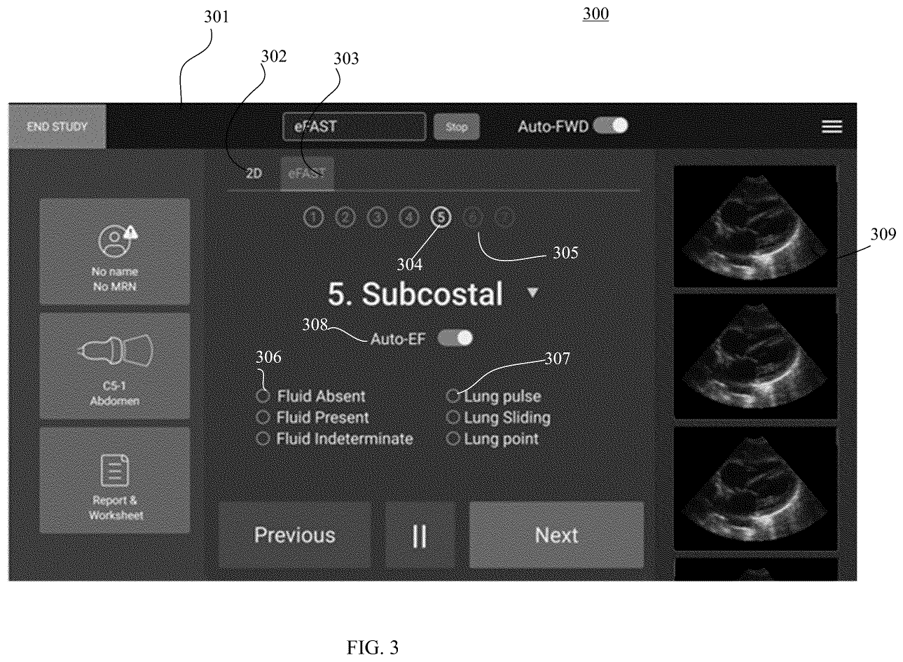

FIG. 3 is a view 300 illustrating an example interface 301 for a protocol self-report step implemented by an ultrasound system according to some embodiments. As shown in FIG. 3, interface 301 includes a plurality of tabs that are associated with corresponding protocols, such as a tab 302 and a tab 303. Some example protocols include FAST, eFAST, BLUE, FATE, MSK ultrasound, or other medical protocols. However, the techniques disclosed herein are not limited to use with these example protocols.

As shown in FIG. 3, tab 302 represents a 2D imaging protocol and tab 303 represents an eFAST protocol. As shown in FIG. 3, tab 303 is selected. Interface 301 includes a plurality of indicators representing steps 1, 2, 3, 4, 5, 6 and 7 of a protocol, such as an indicator 304 representing a step 5 of a protocol and an indicator 305 representing a step 6 of the protocol. In some embodiments, each step of the protocol is customizable. As shown in FIG. 3, step 5 that is associated with a subcostal view of the eFAST protocol is selected by a user as a current protocol step. Interface 301 includes one or more controls, such as a control 306, a control 307 and a control 308 to select auto-populate (self-report) features that are related to the selected protocol step. In some embodiments, the one or more controls are radio buttons, check boxes, or other controls that are selectable by a user. As shown in FIG. 3, control 306 is used to select/unselect a self-report feature (“Fluid Absent”), control 307 is used to select/unselect a self-report feature (“Lung Pulse”). For example, if a Fluid Absent feature is selected, a corresponding field of the worksheet is populated to indicate that free fluid is not detected/observed. In some embodiments, control 308 is used to enable/disable an automatic ejection fraction (“Auto-EF”) measurement. In some embodiments, if the auto-EF measurement is enabled, a corresponding field of the worksheet can be populated to indicate that the automatic ejection fraction measurement is performed. As shown in FIG. 3, each of the self-report features is customizable by being selected/unselected via the controls 306, 307, and 308 on the interface 301 by a user. As shown in FIG. 3, the control 308 indicating auto-EF for the subcostal imaging of the eFAST protocol is turned ON. Interface 301 includes one or more images 309 that are related to the selected protocol step 5.

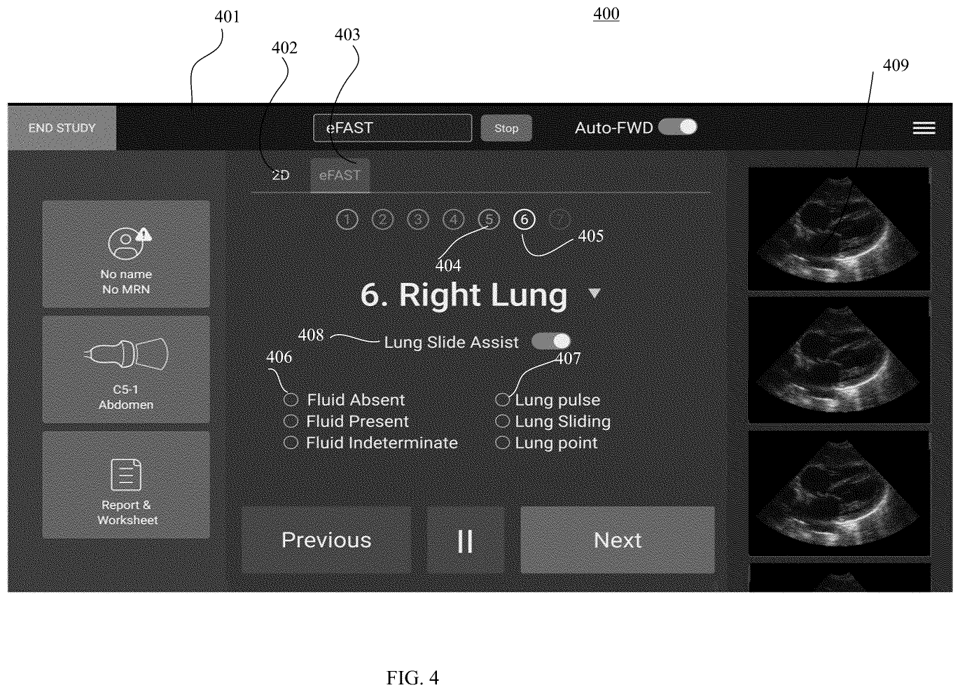

FIG. 4 is a view 400 illustrating an example interface 401 for a protocol self-report step implemented by an ultrasound system according to some embodiments. As shown in FIG. 4, interface 401 includes a plurality of tabs that are associated with corresponding protocols, such as a tab 402 and a tab 403, as described above with respect to FIG. 3. As shown in FIG. 4, tab 403 representing an eFAST protocol is selected. Interface 401 includes a plurality of indicators representing steps 1, 2, 3, 4, 5, 6 and 7 of the eFAST protocol, such as an indicator 404 representing a step 5 of the protocol and an indicator 405 representing a step 6 of the protocol. In some embodiments, each step of the protocol is customizable. As shown in FIG. 4 at 405, step 6 that is associated with a right lung imaging of the eFAST protocol is selected by a user as a current protocol step. Interface 401 includes one or more controls, such as controls 406, 407 and 408 to select self-report features that are related to the selected protocol step, as described above. As shown in FIG. 4, control 406 is used to select/unselect a self-report feature (“Fluid Absent”), control 407 is used to select/unselect a self-report feature (“Lung Pulse”) and control 408 is used to select/unselect a self-report feature (“Lung Slide Assist”). As shown in FIG. 4, each of the self-report features is customizable by being selected/unselected via controls 406, 407 and 408 on interface 401. As shown in FIG. 4, the control 408 indicating lung slide assist for the right lung imaging of the eFAST protocol is turned ON. In an example, the lung slide assist can implement a neural network to determine if lung sliding exists for the right lung selected at step 6 and indicated by indicator 405. In some embodiments, control 408 is used to enable a pneumothorax determination. In some embodiments, if the pneumothorax determination is enabled, a corresponding field of the worksheet can be populated to indicate that the pneumothorax determination is performed. Interface 401 includes one or more images 409 that are related to the selected protocol step 6.

The inclusion of the self-report feature is an advantage over conventional systems that merely display the worksheet, such as the worksheet in its entirety, rather than the specific sections of the worksheet that are related to the protocol step as described herein. For example, in conventional systems, additional worksheet data is displayed that is not relevant to the current protocol step, cluttering the interface and possibly confusing the operator.

In some embodiments, the system implements a neural network to determine an anatomy that is being imaged, and displays an interface for a protocol step based on the anatomy. For instance, the neural network can process an ultrasound image to determine a lung is being imaged and automatically display the subcostal interface illustrated in FIG. 3. In some embodiments, the system can determine an anatomy being imaged with a neural network implemented at least partially in hardware of a computing device (e.g., an ultrasound machine or display device coupled to the ultrasound machine). In some embodiments, the system can then display, automatically and based on the anatomy, a protocol step that includes a question about the anatomy.

In some embodiments, the system determines the identification of an operator of a computing device during an ultrasound examination and then determines, based on the operator identification, an order for a protocol. In some embodiments, the system can then display, during the ultrasound examination, steps of the protocol in the order. For example, the system can learn the order that the user usually does the exam, and then present the protocol steps based on the order associated with the user. As an example, the system can display the interface for step 5 (subcostal view) of the protocol as illustrated in FIG. 3 based on a previous step performed by the user, such as the user performing step 1 or step 6 of the protocol prior to step 5.

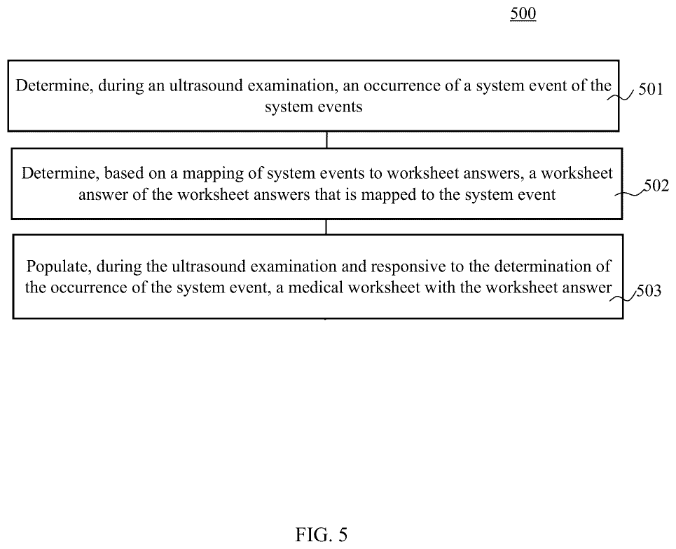

FIG. 5 is a data flow diagram of a process 500 implemented by an ultrasound reporting system according to some embodiments. An ultrasound reporting system can include a memory configured to maintain a mapping of system events to worksheet answers and a processor system coupled to the memory and configured to implement a reporting application at least partially in hardware to perform process 500. In some embodiments, the process 500 is performed using processing logic. The processing logic may comprise hardware (circuitry, dedicated logic, etc.), software (such as is run on a general-purpose computer system or a dedicated machine), firmware, or combinations thereof. At block 501, during an ultrasound examination, an occurrence of a system event of the system events is determined.

Referring to FIG. 5, at block 502, the ultrasound reporting system determines a worksheet answer of the worksheet answers that is mapped to the system event and the determination is based on the mapping of the system events to worksheet answers. At block 503, during the ultrasound examination and responsive to the determination of the occurrence of the system event, the ultrasound reporting system populates a medical worksheet with the worksheet answer. In some embodiments, the system event includes the saving of an ultrasound image, and the worksheet answer includes an affirmative response as to whether the ultrasound image includes an acceptable view. In some embodiments, the mapping of the system event is user-defined and includes an indication that a worksheet question has been answered by the worksheet answer.

In some embodiments, the ultrasound reporting system includes a neural network coupled to the processor system and is implemented at least partially in the hardware to determine, during the ultrasound examination, that the ultrasound examination implements a step of a protocol. The ultrasound reporting system can include a display device coupled to the processor system and implemented to display, automatically and without user intervention, a portion of the medical worksheet that corresponds to a step of the protocol. In some embodiments, the display device displays the portion of the medical worksheet that corresponds to the step of the protocol while not displaying other portions of the medical worksheet that do not correspond to that step of the protocol.

In some embodiments, the ultrasound reporting system includes a display device coupled to the processor system and the memory, and the memory is implemented to maintain a protocol. The processor system is configured to implement the reporting application to determine a next protocol step of the protocol based on the occurrence of the system event corresponding to a current protocol step of the protocol. In some embodiments, the display device is implemented to display a portion of the medical worksheet that corresponds to the next protocol step. In some embodiments, the memory maintains the order of steps of the protocol based on operator identifications. In some embodiments, the reporting application determines an operator identification for an operator performing the ultrasound examination. In some embodiments, the reporting application determines the next protocol step based on the operator identification, the current protocol step, and the orders of steps of the protocol maintained by the memory.

In some embodiments, the ultrasound reporting system includes an ultrasound machine implemented to perform the ultrasound examination. The memory is implemented to maintain orders of steps of a protocol based on operator identifications. The reporting application is implemented to determine an operator identification for an operator of the ultrasound machine. The reporting application is implemented to determine based on the operator identification and the orders of steps of the protocol, a step in the protocol. The reporting application is implemented to configure the ultrasound machine for the step in the protocol. In some embodiments, the reporting application is implemented to receive a user selection to enable an auto-populate feature, and the system populates the medical worksheet based on the user selection. In some embodiments, the memory is implemented to maintain a protocol for the ultrasound examination. The reporting application is implemented to obtain the protocol from an ultrasound machine.

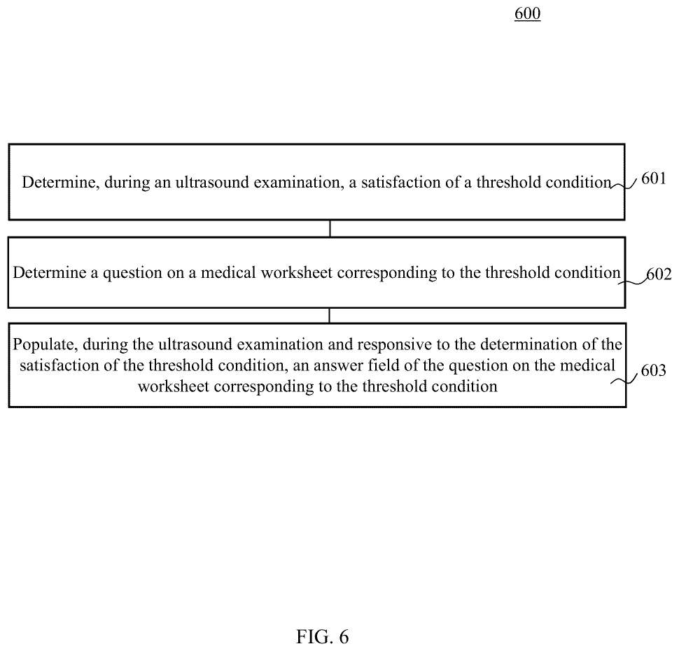

FIG. 6 is a data flow diagram of a process 600 implemented by an ultrasound reporting system according to some embodiments. An ultrasound reporting system can include a processing system and at least one computer-readable storage medium configured to store instructions executable via the processing system to implement a reporting application to perform process 600. In some embodiments, the process 600 is performed using processing logic. The processing logic may comprise hardware (circuitry, dedicated logic, etc.), software (such as is run on a general-purpose computer system or a dedicated machine), firmware, or combinations thereof.

Referring to FIG. 6, at block 601, the ultrasound reporting system determines a satisfaction of a threshold condition during an ultrasound examination. At block 602, the ultrasound reporting system determines a question on a medical worksheet corresponding to the threshold condition, and at block 603, during the ultrasound examination and responsive to the determination of the satisfaction of the threshold condition, the ultrasound reporting system populates an answer field of the question on the medical worksheet corresponding to the threshold condition. In some embodiments, the threshold condition includes a threshold amount of free fluid, and the answer field includes an affirmation of free fluid detection (e.g., an indication that free fluid has been detected). In some embodiments, the threshold condition includes an image quality, and the answer field includes at least one of: an image quality score, an indication of an anatomy in an ultrasound image, and an indication of an interventional instrument in the ultrasound image.

In some embodiments, at least one computer-readable storage medium is implemented to maintain a user-defined mapping of threshold conditions to questions on the medical worksheet, and the determination of the question is based on the user-defined mapping. In some embodiments, the ultrasound examination includes a protocol, the determination of the satisfaction of the threshold condition is performed during a current step of the protocol, and the reporting application is implemented to determine the question as part of the current step of the protocol. In some embodiments, the ultrasound examination includes a protocol, the determination of the satisfaction of the threshold condition is performed during a current step of the protocol, and the reporting application is implemented to determine the question as part of a protocol step different from the current step of the protocol.

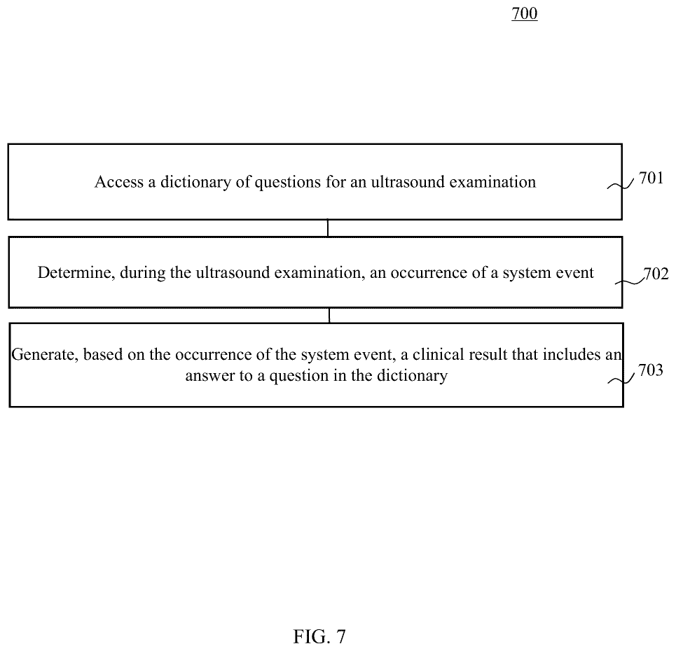

FIG. 7 is a data flow diagram of a process 700 implemented by an ultrasound reporting system according to some embodiments. An ultrasound reporting system can include a processing system and at least one computer-readable storage medium configured to store instructions executable via the processing system to implement a reporting application to perform process 700. In some embodiments, the process 700 is performed using processing logic. The processing logic may comprise hardware (circuitry, dedicated logic, etc.), software (such as is run on a general-purpose computer system or a dedicated machine), firmware, or combinations thereof.

Referring to FIG. 7, at block 701, the ultrasound reporting system accesses a dictionary of questions for an ultrasound examination. At block 702, during the ultrasound examination, the ultrasound reporting system determines an occurrence of a system event. At block 703, the ultrasound reporting system generates, based on the occurrence of the system event, a clinical result that includes an answer to a question in the dictionary. In some embodiments, the reporting system uses a reporting application to populate a medical worksheet with the clinical result. In some embodiments, the reporting application is implemented to enter the clinical result into an application protocol interface (API), and populate, from the API, the medical worksheet with the clinical result.

In some embodiments, the reporting application is implemented to receive a dictionary update. The reporting application is implemented to amend, based on the dictionary update, the dictionary. In some embodiments, amending the dictionary includes an action selected from the group consisting of to add at least one question from the dictionary and to remove at least one additional question from the dictionary.

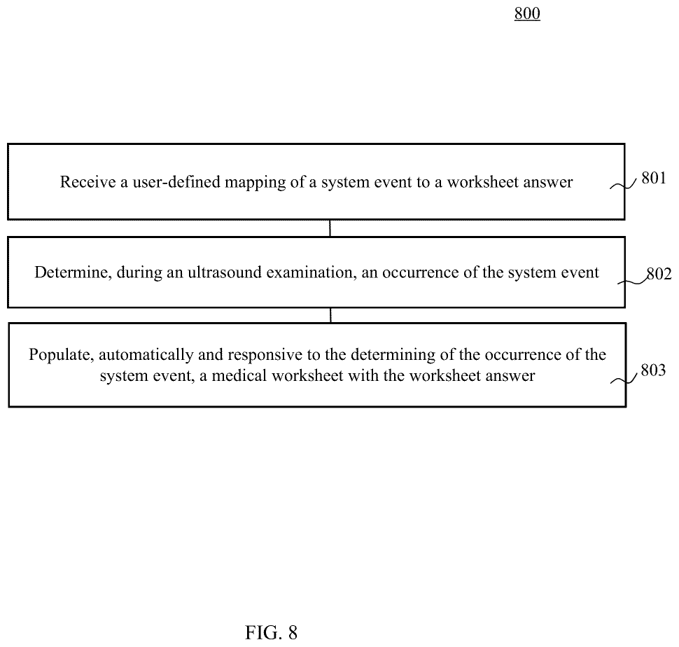

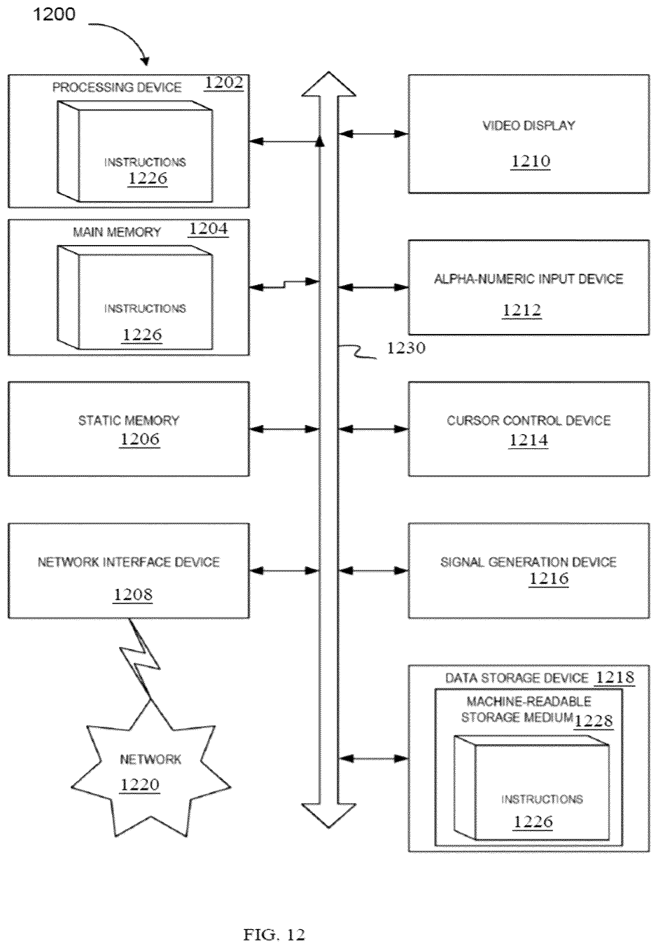

FIG. 8 is a data flow diagram of a method 800 implemented by a computing device according to some embodiments. In some embodiments, the computing device includes processing logic that can include hardware (e.g., circuitry, dedicated logic, memory, etc.), software (such as is run on a general-purpose computer system or a dedicated machine), firmware (e.g., software programmed into a read-only memory), or combinations thereof. In some embodiments, the process is performed by one or more processors of a computing device such as, for example, but not limited to, an ultrasound machine with an ultrasound imaging subsystem. In some embodiments, the computing device is represented by a computing device as shown in FIG. 12.

Referring to FIG. 8, the method 800 includes receiving a user-defined mapping of a system event to a worksheet answer at block 801 and determining, during an ultrasound examination, an occurrence of the system event at block 802. The method 800 also includes populating, automatically and responsive to the determining of the occurrence of the system event, a medical worksheet with the worksheet answer at block 803. In some embodiments, the system event includes the saving of an ultrasound image. In some embodiments, the worksheet answer includes an affirmative response to whether the ultrasound image includes an acceptable view. In some embodiments, the user-defined mapping includes a worksheet question answered by the worksheet answer. In some embodiments, the method 800 includes determining, with a neural network implemented at least partially in hardware of the computing device, that the ultrasound examination includes a step of a protocol.

In some embodiments, the method 800 includes displaying, automatically and without user intervention, a portion of the medical worksheet that corresponds to the step of the protocol. In some embodiments, determining that the ultrasound examination includes the step of the protocol includes determining an anatomy being imaged. In some embodiments, the method 800 includes determining a next protocol step for the ultrasound examination, based on the occurrence of the system event. In some embodiments, the method 800 includes displaying a portion of the medical worksheet that corresponds to the next protocol step. In some embodiments, the method 800 includes determining an operator identification that identifies an operator of the computing device. In some embodiments, determining the next protocol step is based on the operator identification.

In some embodiments, the method 800 includes determining an operator identification that identifies an operator of the computing device. In some embodiments, the method 800 includes obtaining, based on the operator identification, the user-defined mapping from a database of user-defined mappings. In some embodiments, the method 800 includes obtaining, based on the operator identification, the medical worksheet from the database.

In some embodiments, the method 800 includes receiving a user selection of a starting point in a protocol. In some embodiments, the method 800 includes configuring, based on the starting point in the protocol, the computing device for the ultrasound examination. In some embodiments, the method 800 includes receiving a user selection of an auto-populate feature. In some embodiments, populating the medical worksheet with the worksheet answer is based on the receiving the user selection. In some embodiments, the method 800 includes receiving a user selection of a worksheet display feature. In some embodiments, the method 800 includes displaying, based on the user selection, the medical worksheet.

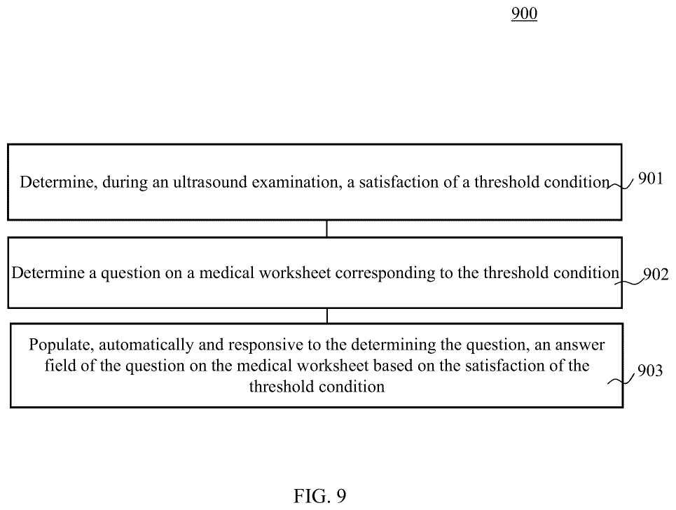

FIG. 9 is a data flow diagram of a method 900 implemented by a computing device according to some embodiments. In some embodiments, the computing device includes processing logic that can include hardware (e.g., circuitry, dedicated logic, memory, etc.), software (such as is run on a general-purpose computer system or a dedicated machine), firmware (e.g., software programmed into a read-only memory), or combinations thereof. In some embodiments, the process is performed by one or more processors of a computing device such as, for example, but not limited to, an ultrasound machine with an ultrasound imaging subsystem. In some embodiments, the computing device is represented by a computing device as shown in FIG. 12.

Referring to FIG. 9, the method 900 includes determining, during an ultrasound examination, a satisfaction of a threshold condition at block 901. The method 900 also includes determining a question on a medical worksheet corresponding to the threshold condition at block 902. The method 900 further includes populating, automatically and responsive to the determining the question, an answer field of the question on the medical worksheet based on the satisfaction of the threshold condition at block 903. In some embodiments, the threshold condition includes a threshold amount of free fluid and the answer field includes an affirmation of free fluid detection. In some embodiments, the threshold condition includes an image quality, and the answer field includes at least one of an image quality score, an indication of an anatomy in an ultrasound image, and an indication of an interventional instrument in the ultrasound image. In some embodiments, the computing device implements at least one neural network to perform at least one of: determining the satisfaction of the threshold condition, determining the question on the medical worksheet corresponding to the threshold condition, and populating the answer field. In some embodiments, determining the question corresponding to the threshold condition includes determining that the question can be answered by the satisfaction of the threshold condition. In some embodiments, the computing device determines the question corresponding to the threshold condition based on a user-defined mapping of the satisfaction of the threshold condition to the question and the answer field.

In some embodiments, the ultrasound examination includes a protocol, determining the satisfaction of the threshold condition is performed during a current step of the protocol, and determining the question includes determining the question as part of the current step of the protocol. In some embodiments, the ultrasound examination includes a protocol, the determining the satisfaction of the threshold condition is performed during a current step of the protocol, and the determining the question includes determining the question as part of a subsequent step of the protocol.

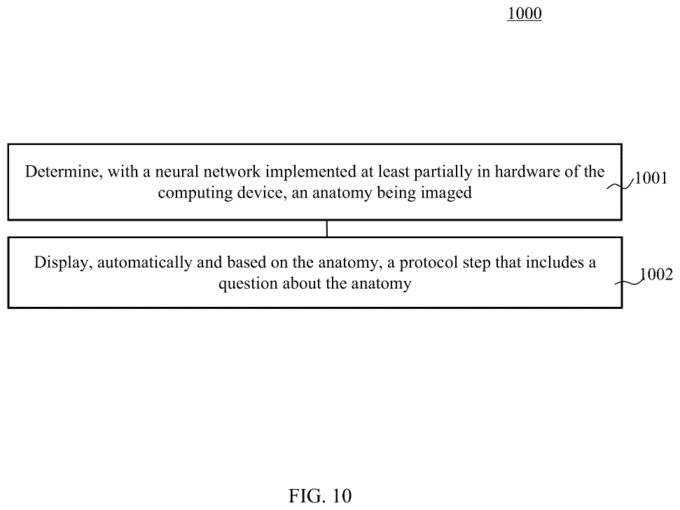

FIG. 10 is a data flow diagram of a method 1000 implemented by a computing device according to some embodiments. In some embodiments, the computing device includes processing logic that can include hardware (e.g., circuitry, dedicated logic, memory, etc.), software (such as is run on a general-purpose computer system or a dedicated machine), firmware (e.g., software programmed into a read-only memory), or combinations thereof. In some embodiments, the process is performed by one or more processors of a computing device such as, for example, but not limited to, an ultrasound machine with an ultrasound imaging subsystem. In some embodiments, the computing device is represented by a computing device as shown in FIG. 12.

Referring to FIG. 10, the method 1000 includes determining, with a neural network implemented at least partially in hardware of the computing device, an anatomy being imaged at block 1001. The method 1000 also includes displaying, automatically and based on the anatomy, a protocol step that includes a question about the anatomy at block 1002.

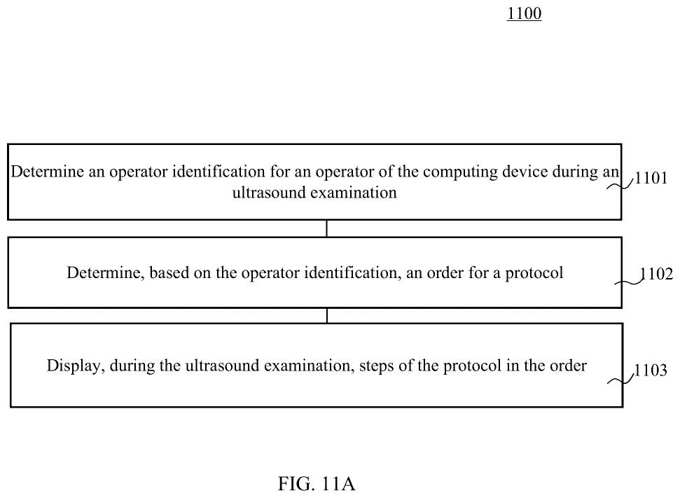

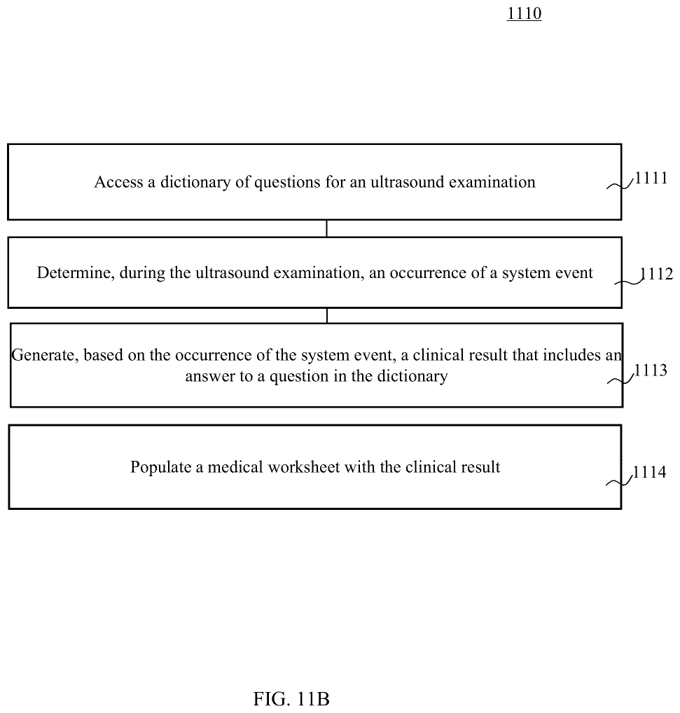

FIG. 11A is a data flow diagram of a method 1100 implemented by a computing device according to some embodiments. In some embodiments, the computing device includes processing logic that can include hardware (e.g., circuitry, dedicated logic, memory, etc.), software (such as is run on a general-purpose computer system or a dedicated machine), firmware (e.g., software programmed into a read-only memory), or combinations thereof. In some embodiments, the process is performed by one or more processors of a computing device such as, for example, but not limited to, an ultrasound machine with an ultrasound imaging subsystem. In some embodiments, the computing device is represented by a computing device as shown in FIG. 12.

Referring to FIG. 11A, the method 1100 includes determining an operator identification for an operator of the computing device during an ultrasound examination at block 1101. The method 1100 includes determining, based on the operator identification, an order for a protocol at block 1102. The method 1100 also includes displaying, during the ultrasound examination, steps of the protocol in the order at block 1103.

FIG. 11B is a data flow diagram of a method 1110 implemented by a computing device according to some embodiments. In some embodiments, the computing device includes processing logic that can include hardware (e.g., circuitry, dedicated logic, memory, etc.), software (such as is run on a general-purpose computer system or a dedicated machine), firmware (e.g., software programmed into a read-only memory), or combinations thereof. In some embodiments, the process is performed by one or more processors of a computing device such as, for example, but not limited to, an ultrasound machine with an ultrasound imaging subsystem. In some embodiments, the computing device is represented by a computing device as shown in FIG. 12.

Referring to FIG. 11B, the method 1110 includes accessing a dictionary of questions for an ultrasound examination at block 1111. The method 1110 includes determining, during the ultrasound examination, an occurrence of a system event at block 1112. The method 1110 includes generating, based on the occurrence of the system event, a clinical result that includes an answer to a question in the dictionary at block 1113. The method 1110 includes populating a medical worksheet with the clinical result at block 1114. In some embodiments, the medical worksheet corresponds to a protocol for the ultrasound examination. In some embodiments, the method 1110 includes entering the clinical result into an application protocol interface (API) and populating, from the API, a medical worksheet with the clinical result. In some embodiments, the method 1110 includes receiving a dictionary update and updating, based on the dictionary update, the dictionary with a new question. In some embodiments, the method 1110 includes receiving a dictionary update and updating, based on the dictionary update, the dictionary by removing at least one question from the dictionary.

Embodiments of the systems, devices, and methods for automated reporting in POCUS workflows disclosed herein provide numerous advantages over conventional systems. Embodiments disclosed herein provide automated and/or assisted reporting that extends the general artificial intelligence (AI) initiative of simplifying required tasks and immediate data entry within the clinical context. Embodiments disclosed herein also provide in context a checklist of reporting clinical observations and support various standards of care via customization of protocols for worksheet content.

FIG. 12 is a block diagram of an example computing device 1200 that may perform one or more of the operations described herein, in accordance with some embodiments. Computing device 1200 may be connected to other computing devices in a LAN, an intranet, an extranet, and/or the Internet. The computing device may operate in the capacity of a server machine in client-server network environment or in the capacity of a client in a peer-to-peer network environment. The computing device may be provided by a personal computer (PC), a server computing, a desktop computer, a laptop computer, a tablet computer, a smartphone, or any machine capable of executing a set of instructions (sequential or otherwise) that specify actions to be taken by that machine. Further, while only a single computing device is illustrated, the term “computing device” shall also be taken to include any collection of computing devices that individually or jointly execute a set (or multiple sets) of instructions to perform the methods and processes discussed herein. In some embodiments, the computing device 1200 may be one or more of an access point and a packet forwarding component.

The example computing device 1200 may include a processing device (e.g., a general-purpose processor, a PLD, etc.) 1202, a main memory 1204 (e.g., synchronous dynamic random-access memory (DRAM), read-only memory (ROM)), a static memory 1206 (e.g., flash memory and a data storage device 1218), which may communicate with each other via a bus 1230. Processing device 1202 may be provided by one or more general-purpose processing devices such as a microprocessor, central processing unit, or the like. In an illustrative example, processing device 1202 may comprise a complex instruction set computing (CISC) microprocessor, reduced instruction set computing (RISC) microprocessor, very long instruction word (VLIW) microprocessor, or a processor implementing other instruction sets or processors implementing a combination of instruction sets. Processing device 1202 may also comprise one or more special-purpose processing devices such as an application specific integrated circuit (ASIC), a field programmable gate array (FPGA), a digital signal processor (DSP), network processor, or the like. The processing device 1202 may be configured to execute the operations described herein, in accordance with one or more aspects of the present disclosure, for performing the operations and steps discussed herein.

Computing device 1200 may further include a network interface device 1208 which may communicate with a network 1220. The computing device 1200 also may include a video display unit 1210 (e.g., a liquid crystal display (LCD) or a cathode ray tube (CRT)), an alphanumeric input device 1212 (e.g., a keyboard), a cursor control device 1214 (e.g., a mouse) and an acoustic signal generation device 1216 (e.g., a speaker, and/or a microphone). In one embodiment, video display unit 1210, alphanumeric input device 1212, and cursor control device 1214 may be combined into a single component or device (e.g., an LCD touch screen).

Data storage device 1218 may include a computer-readable storage medium 1228 on which may be stored one or more sets of instructions 1226, e.g., instructions for carrying out the operations described herein, in accordance with one or more aspects of the present disclosure. For instance, the instructions 1226 can implement the reporting application, as described herein. Instructions 1226 may also reside, completely or at least partially, within main memory 1204 and/or within processing device 1202 during execution thereof by computing device 1200, main memory 1204 and processing device 1202 also constituting computer-readable media. The instructions may further be transmitted or received over a network 1220 via network interface device 1208.

While computer-readable storage medium 1228 is shown in an illustrative example to be a single medium, the term “computer-readable storage medium” should be taken to include a single medium or multiple media (e.g., a centralized or distributed database and/or associated caches and servers) that store the one or more sets of instructions. In some embodiments, the computer-readable storage medium 1228 implements the database of user-defined mappings, as described above. The term “computer-readable storage medium” shall also be taken to include any medium that is capable of storing, encoding or carrying a set of instructions for execution by the machine and that cause the machine to perform the methods described herein. The term “computer-readable storage medium” shall accordingly be taken to include, but not be limited to, solid-state memories, optical media and magnetic media.

Unless specifically stated otherwise, terms such as “transmitting,” “determining,” “receiving,” “generating,” “or the like, refer to actions and processes performed or implemented by computing devices that manipulates and transforms data represented as physical (electronic) quantities within the computing device's registers and memories into other data similarly represented as physical quantities within the computing device memories or registers or other such information storage, transmission or display devices. Also, the terms “first,” “second,” “third,” “fourth,” etc., as used herein are meant as labels to distinguish among different elements and may not necessarily have an ordinal meaning according to their numerical designation.

Examples described herein also relate to an apparatus for performing the operations described herein. This apparatus may be specially constructed for the required purposes, or it may comprise a general-purpose computing device selectively programmed by a computer program stored in the computing device. Such a computer program may be stored in a computer-readable non-transitory storage medium, such as a storage memory.

The methods and illustrative examples described herein are not inherently related to any particular computer or other apparatus. Various general-purpose systems may be used in accordance with the teachings described herein, or it may prove convenient to construct more specialized apparatus to perform the required method steps. The required structure for a variety of these systems will appear as set forth in the description above.

The above description is intended to be illustrative, and not restrictive. Although the present disclosure has been described with references to specific illustrative examples, it will be recognized that the present disclosure is not limited to the examples described. The scope of the disclosure should be determined with reference to the following claims, along with the full scope of equivalents to which the claims are entitled.

As used herein, the singular forms “a”, “an” and “the” are intended to include the plural forms as well, unless the context clearly indicates otherwise. It will be further understood that the terms “comprises”, “comprising”, “includes”, and/or “including”, when used herein, specify the presence of stated features, integers, steps, operations, elements, and/or components, but do not preclude the presence or addition of one or more other features, integers, steps, operations, elements, components, and/or groups thereof. Therefore, the terminology used herein is for the purpose of describing particular embodiments only and is not intended to be limiting.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Although the method operations were described in a specific order, it should be understood that other operations may be performed in between described operations, described operations may be adjusted so that they occur at slightly different times or the described operations may be distributed in a system which allows the occurrence of the processing operations at various intervals associated with the processing.

Various units, circuits, or other components may be described or claimed as “configured to” or “configurable to” perform a task or tasks. In such contexts, the phrase “configured to” or “configurable to” is used to connote structure by indicating that the units/circuits/components include structure (e.g., circuitry) that performs the task or tasks during operation. As such, the unit/circuit/component can be said to be configured to perform the task, or configurable to perform the task, even when the specified unit/circuit/component is not currently operational (e.g., is not on). The units/circuits/components used with the “configured to” or “configurable to” language include hardware—for example, circuits, memory storing program instructions executable to implement the operation, etc. Reciting that a unit/circuit/component is “configured to” perform one or more tasks, or is “configurable to” perform one or more tasks, is expressly intended not to invoke 35 U.S.C. 112, sixth paragraph, for that unit/circuit/component.

Additionally, “configured to” or “configurable to” can include generic structure (e.g., generic circuitry) that is manipulated by software and/or firmware (e.g., an FPGA or a general-purpose processor executing software) to operate in manner that is capable of performing the task(s) at issue. “Configured to” may also include adapting a manufacturing process (e.g., a semiconductor fabrication facility) to fabricate devices (e.g., integrated circuits) that are adapted to implement or perform one or more tasks. “Configurable to” is expressly intended not to apply to blank media, an unprogrammed processor or unprogrammed generic computer, or an unprogrammed programmable logic device, programmable gate array, or other unprogrammed device, unless accompanied by programmed media that confers the ability to the unprogrammed device to be configured to perform the disclosed function(s).

Reference in the specification to “one embodiment”, “an embodiment”, “one example”, or “an example” means that a particular feature, structure, or characteristic described in conjunction with the embodiment can be included in at least one embodiment. The appearances of the phrases “in one embodiment” or “in an embodiment” in various places in the specification do not necessarily all refer to the same embodiment. The processes depicted in the figures that follow are performed by processing logic that comprises hardware (e.g., circuitry, dedicated logic, etc.), software, or a combination of both. Although the processes are described below in terms of some sequential operations, it should be appreciated that some of the operations described may be performed in a different order. Moreover, some operations may be performed in parallel rather than sequentially.

In the specification, the term “and/or” describes three relationships between objects that may exist. For example, A and/or B may represent the following cases: only A exists, both A and B exist, and only B exist, where A and B may be singular or plural.

The foregoing description, for the purpose of explanation, has been described with reference to specific embodiments. However, the illustrative discussions above are not intended to be exhaustive or to limit embodiments of the invention to the precise forms disclosed. Many modifications and variations are possible in view of the above teachings. The embodiments were chosen and described in order to best explain the principles of the embodiments and its practical applications, to thereby enable others skilled in the art to best utilize the embodiments and various modifications as may be suited to the particular use contemplated. Accordingly, the present embodiments are to be considered as illustrative and not restrictive, and the invention is not to be limited to the details given herein, but may be modified within the scope and equivalents of the appended claims.

What is claimed is:

1. An ultrasound reporting system comprising:
a memory configured to maintain a mapping of system events to worksheet answers; and
a processor system configured to implement a reporting application at least partially in hardware, the reporting application implemented to:
determine, during an ultrasound examination, an occurrence of a system event of the system events;
determine, based on the mapping, a worksheet answer of the worksheet answers that is mapped to the system event; and
populate, during the ultrasound examination and responsive to the determination of the occurrence of the system event, a medical worksheet with the worksheet answer.

2. The ultrasound reporting system as described in claim 1, wherein the system event includes saving an ultrasound image, and the worksheet answer includes an affirmative response that the ultrasound image includes an acceptable view.

3. The ultrasound reporting system as described in claim 1, wherein the mapping of the system event is user-defined and includes a worksheet question answered by the worksheet answer.

4. The ultrasound reporting system as described in claim 1, further comprising:
a neural network implemented at least partially in the hardware to determine, during the ultrasound examination, that the ultrasound examination implements a step of a protocol; and
a display device implemented to display, automatically and without user intervention, a portion of the medical worksheet that corresponds to the step of the protocol.

5. The ultrasound reporting system as described in claim 4, wherein the display device is implemented to display the portion of the medical worksheet that corresponds to the step of the protocol including not to display other portions of the medical worksheet that do not correspond to the step of the protocol.

6. The ultrasound reporting system as described in claim 1, further comprising a display device, wherein the memory is implemented to maintain a protocol, the reporting application is implemented to determine, based on the occurrence of the system event corresponding to a current protocol step of the protocol, a next protocol step of the protocol, and the display device is implemented to display a portion of the medical worksheet that corresponds to the next protocol step.

7. The ultrasound reporting system as described in claim 6, wherein the memory is implemented to maintain orders of steps of the protocol based on operator identifications, the reporting application is implemented to determine an operator identification for an operator performing the ultrasound examination, and the determination of the next protocol step is based on the operator identification, the current protocol step, and the orders of steps of the protocol maintained by the memory.

8. The ultrasound reporting system as described in claim 1, further comprising an ultrasound machine implemented to perform the ultrasound examination, wherein the memory is implemented to maintain orders of steps of a protocol based on operator identifications, wherein the reporting application is implemented to:
determine an operator identification for an operator of the ultrasound machine;
determine, based on the operator identification and the orders of steps of the protocol, a step in the protocol; and
configure the ultrasound machine for the step in the protocol.

9. The ultrasound reporting system as described in claim 1, wherein the reporting application is implemented to receive a user selection to enable an auto-populate feature, and said populate is based on the user selection.

10. The ultrasound reporting system as described in claim 1, wherein the memory is implemented to maintain a protocol for the ultrasound examination, and the reporting application is implemented to obtain the protocol from an ultrasound machine.

11. An ultrasound reporting system comprising:
a processing system; and
at least one computer-readable storage medium configured to store instructions executable via the processing system to implement a reporting application configured to:
determine, during an ultrasound examination, a satisfaction of a threshold condition;
determine a question on a medical worksheet corresponding to the threshold condition; and
populate, during the ultrasound examination and responsive to the determination of the satisfaction of the threshold condition, an answer field of the question on the medical worksheet corresponding to the threshold condition.

12. The ultrasound reporting system as described in claim 11, wherein the threshold condition includes a threshold amount of free fluid, and the answer field includes an affirmation of free fluid detection.

13. The ultrasound reporting system as described in claim 11, wherein the threshold condition includes an image quality, and the answer field includes at least one of an image quality score, an indication of an anatomy in an ultrasound image, and an indication of an interventional instrument in the ultrasound image.

14. The ultrasound reporting system as described in claim 11, wherein the at least one computer-readable storage medium is implemented to maintain a user-defined mapping of threshold conditions to questions on the medical worksheet, and the determination of the question is based on the user-defined mapping.

15. The ultrasound reporting system as described in claim 11, wherein the ultrasound examination includes a protocol, the determination of the satisfaction of the threshold condition is during a current step of the protocol, and the reporting application is implemented to determine the question as part of the current step of the protocol.

16. The ultrasound reporting system as described in claim 11, wherein the ultrasound examination includes a protocol, the determination of the satisfaction of the threshold condition is during a current step of the protocol, and the reporting application is implemented to determine the question as part of a protocol step different from the current step of the protocol.

17. An ultrasound reporting system comprising:
a processing system; and
at least one computer-readable storage medium configured to store instructions executable via the processing system to implement a reporting application configured to:
access a dictionary of questions for an ultrasound examination;
determine, during the ultrasound examination, an occurrence of a system event; and
generate, based on the occurrence of the system event, a clinical result that includes an answer to a question in the dictionary.

18. The ultrasound reporting system as described in claim 17, wherein the reporting application is implemented to populate a medical worksheet with the clinical result.

19. The ultrasound reporting system as described in claim 18, wherein the reporting application is implemented to enter the clinical result into an application protocol interface (API), and populate, from the API, the medical worksheet with the clinical result.

20. The ultrasound reporting system as described in claim 17, wherein the reporting application is implemented to:
receive a dictionary update; and
amend, based on the dictionary update, the dictionary, said amend including an action selected from the group consisting of to add at least one question from the dictionary and to remove at least one additional question from the dictionary.
